(19) United States
(12) Patent Application Publication
(10) Pub. No.: US 2009/0035244 A1
(43) Pub. Date: Feb. 5, 2009

(54) COSMETIC COMPOSITION COMPRISING FUNCTIONALISED POLY(ALPHA OLEFIN-COPOLYMER-MALEIC ANHYDRIDE)

(75) Inventors: Pietro Rando, Opera (IT); Giuseppe Maio, Zelo Surrigone (IT)

Correspondence Address: AKERMAN SENTERFITT, P.O. BOX 3188, WEST PALM BEACH, FL, 33402-3188, US

(73) Assignee: Intercos S.p.A, Milano (IT)

(21) Appl. No.: 12/280,250

(22) PCT Filed: Feb. 21, 2007
(86) PCT No.: PCT/EP2007/051697

§ 371 Date: Aug. 21, 2008

(30) Foreign Application Priority Data
Feb. 23, 2006 (IT) MI2006A000326

(51) Int. Cl. A61K 8/81; C08F 222/06; A61Q 1/12; A61Q 1/06; A61Q 1/10

(52) U.S. Cl. 424/64; 424/78.03; 526/271; 424/69; 424/70.7

(57) ABSTRACT

A cosmetic composition is described comprising functionalised polyanhydrides, specifically functionalised poly(alphaolefin-copolymer-maleic anhydride) copolymers, thereafter also indicated as functionalised polyanhydrides. It is further described a process to prepare such a cosmetic composition by the functionalisation of the polyanhydrides by opening reactions of the anhydride ring with molecules containing at least one OH or NH₂ group.

The present invention relates to a cosmetic composition comprising functionalised polymers, specifically functionalised poly(alphaolefins-copolymers-maleic anhydride) copolymers, thereafter also indicated as functionalised polyanhydrides, and a process to functionalise the same.

The use of functionalised polyanhydrides is known in the pharmaceutical field for the controlled and localised drug release.

The use of polyanhydrides as film-forming agents having water-repellent and lasting properties is known in the cosmetic field.

However, it is also known that the contact of these polyanhydrides with the skin or with physiological organic fluids may give rise to undesired opening reactions of the anhydride ring and cause the polyanhydrides to covalently bind to the proteins and other organic compounds forming skin.

Furthermore, there is a special interest in the cosmetic field for the use of the polymers obtained by the functionalisation of polyanhydrides, which could be employed for the sustained release of active compounds or to extend the persistence thereof in situ.

U.S. Pat. No. 6,495,150 describes cosmetic compositions containing monomers of derivatives of maleic anhydride of formula I:

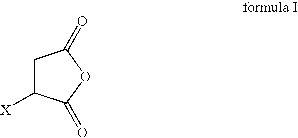

in which X contains a heteroatom and has a bond which may be hydrolysed with the anhydride structure so as to be released in the course of time. X is defined as “Cosmetic benefit agent” (COBA). According to the authors, the anhydride binds to the derma by reaction with the epidermal proteins.

Patent application US 2005/0191249 describes poly(alphaolefins-co-maleic anhydride) of formula II, which are functionalised with synthetic derivatives of crylene or fluorene, which are responsible for the sun filter properties, so as to yield compounds of formula III, in which R³ is an alphaolefin such as styrene, ethylene, isobutene or methylvinylether, and R⁴ is a derivative of crylene or fluorene. Specifically, 2-cyano-3,3-diphenylacrylic acid and (cyano(9H)-fluoren-9-iliden)acetic acid have been used.

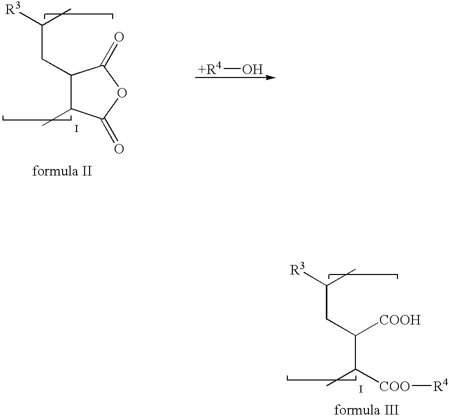

U.S. Pat. No. 6,423,785 describes maleic anhydride copolymers containing amine oxides used to disperse pigments and charges. The comonomers of maleic anhydride employed comprise at least one monomer containing a double vinyl bond, e.g. styrene and derivatives, (met)acrylic acid or esters, alkylvinyl ethers, vinyl acetate, and itaconic esters. The molecular weight of the copolymer varies from 1,000 to 100,000 g/mol. These copolymers are reacted with diamines and then oxidised with the formation of amine oxide groups.

U.S. Pat. No. 6,492,455 uses the reaction product of C₆—C₅₂ alphaolefins, preferably C₆—C₁₈ alphaolefins, with maleic anhydride in aqueous solution, giving rise to products having a molecular weight of 2-5,000 g/mol, which are functionalised by the reaction with secondary or ternary amines for cosmetic applications (such as hair sprays and sun block lotions), or industrial applications (such as ink formulations for ink jet printers).

In view of the described state of the art, it is the object of the present invention to provide a cosmetic composition which promotes the sustained and controlled release of the active compound, confers improved film-forming properties to the colour, enhances the wettability of the pigments, the brightness of the film, the lasting, and has a protective action on the skin.

According to the present invention, such an object is achieved by a cosmetic composition as defined in claim 1.

The Applicant has discovered that the multifunctionalisation of the polyanhydrides with one o more functional molecules having various nature allows to differentiate and render said polyanhydrides employable in any cosmetic composition, especially in make-up products such as foundation, powder, eye-shadows, lipsticks and mascara in the form of loose and cake powders, fluid and cream emulsions, casts, etc.

The molecules employed for the functionalisation in the present invention contain at least one OH or NH₂ group, though not amine oxides, nor secondary or ternary amines. Furthermore, the reagents which confer filtering properties to the cosmetic compositions of the invention are natural and not synthetic organic compounds.

The functionalised polyanhydrides obtained according to the present invention have a high molecular weight, thus conferring to the product containing them a greater brightness and a better adhesion to the derma.

The use of the functionalised polyanhydrides of the present invention allows to exploit:

a) their technological properties to improve the brightness, the affinity for the colour phase and the persistence of the colour film generated after application;

b) the pro-active action on the epidermal physiology;

c) the versatility of the derivatives, by varying the different active compounds which may be inserted in the polymer chain.

Going into greater detail, the cosmetic composition according to the present invention comprises a dispersion in a solvent or reaction medium of a functionalised polymer having a molecular weight between 4,000 and 1,000,000 g/mol or more generally between 500 and 1,000,000 g/mol: the use of these polymer structures gives rise to different opportunities, especially for decorative cosmetics, by combining different performance and conferring film-forming power to the colour, wettability of the pigments, enhancement of the brightness of the film and protection to the skin at the same time, in virtue of the high lipophily of the polymer and also the modulated release of the active compound.

The special architecture of the polymer alternates carboxyl and ester groups with different length alkyl chains (C₂—C₃₄ or C₂—C₃₆) which branch from the central framework, and is also responsible for its marked amphiphilic characteristic. The lipophilic portion ensures comfort and protection during the persistence on the skin. The esteric moiety is formed by repetitive units of single or multiple functional active molecules which may have different performance.

The polymers contained in the composition according to the present invention promote a protective action on the skin and confer a sustained and controlled release of the active compound.

The cosmetic composition contains from 0.5 wt % to 95 wt %, preferably from 10 wt % to 50 wt %, of the dispersion of the functionalised polyanhydride in a solvent or reaction medium.

The weight concentration of said polymer in its reaction medium varies from 5% to 85% or more in general from 1% to 85%, preferably from 15% to 60% of the formula according to the type of functionalisation and to the use it is intended for.

The cosmetic composition of the present invention may further contain solvents, waxes, excipients, dyes, preservatives, (co)polymers, fragrances, perfumes, vitamins, antioxidants, vegetal or mineral oils and fats, gloss agents, surfactants, or other ingredients which are normally used in cosmetics and are included in the CTFA.

Any organic or silicone solvent for cosmetic use may be employed as a solvent. Typical examples are C₈—C₂₄ aliphatic hydrocarbons, e.g. isoparaffins such as isooctane, isononane, isodecane, isododecane, Isopars®; typical examples of silicone solvents are cyclosiloxanes, dimeticones etc.

Candelilla wax, carnauba wax, bees wax, ceresins, microcrystalline waxes, paraffins, silicone waxes, polyethylene waxes etc. may be used as waxes.

Talc, mica, silica, kaolin, zinc oxide, calcium carbonate, magnesium carbonate and phosphate, starch and derivatives, nylon, polyethylene, acrylic (co)polymers, etc. may be used as excipients.

Iron oxides, chromium oxides and/or hydroxides, ultramarine blue and pink, manganese violet, titanium dioxide, glossy pigments on mica substrates or bismuth oxychloride, carmine lakes and organic dyes as listed by the CTFA may be used as dyes.

The appropriately functionalised polyanhydrides may be inserted in any type of cosmetic composition, especially for:

the make-up of lips, as a stick or cream

the make-up of eyes, eye-shadows as a pressed powder or as an emulsion or by casting and mascara

the make-up of the face, pressed powder or emulsion or by casting without excluding other types of cosmetic applications.

The present invention also refers to a process directed to obtain the functionalised poly(alphaolefins-co-maleic anhydride) of formula IV by opening reactions of the anhydride ring of polyanhydrides of formula V with functional molecules containing at least one OH or NH₂ group, in a solvent or reaction medium, as depicted in the following scheme.

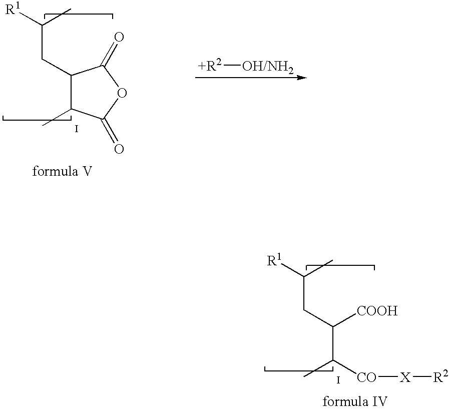

The polyanhydrides of formula V, in which R¹ is a C₂—C₃₄ alkyl, have a molecular weight comprised between 2,000 and 600,000 g/mol or more in general between 350 and 600,000 g/mol.

The functionalisation reaction may be performed by esterification or amidation reactions between molecules respectively containing OH or NH₂ groups and the anhydride functionalities in the presence of a reaction medium and possibly of a catalyst to accelerate times and decrease the reaction temperatures.

Both bases and acids may be used as catalysts, in the examples of the present invention inorganic acids such as hydrochloric acid, perchloric acid, sulphuric acid, or organic acids such as ossalic acid, citric acid, glutammic acid etc. have been specifically used.

Solvents chosen from the group of (C₈—C₂₀) triglycerides, alcohol or polyalcohol esters with fatty acids (such as trimethylol propane triisostearate, octyl stearate), hydrocarbons (such as isododecane and hydrogenated polyolefins), silicones (such as cyclopentasiloxane and dimeticones), organosilicones and water may preferably be used as solvent or reaction medium. The resulting functionalised polyanhydrides are therefore already dispersed in the solvent, ready to use.

The reaction may be performed between room temperature and 150° C.

The course of the reaction is followed by Fourier Transform Infrared Spectrophotometry (FT-IR) in order to monitor the anhydride band at 1709-1790 cm⁻¹ which disappears as the reaction proceeds.

The control on molecular weight is verified by the technique that employs size exclusion chromatography (SEC) or gel permeation chromatography (GPC), (isocratic pump HPLC system, refraction index detector and 40° C. thermocontrolled column system). Specifically, a solution in THF of the sample to be tested is injected in the column; thus the molecular weight curve is extrapolated by means of a special software employing polystyrene standards as a calibration curve.

In a preferred embodiment of the present invention, the reaction product is a dispersion in the solvent or in its reaction medium of the functionalised polyanhydride to be used as such in the cosmetic compositions which are the object of the present invention. Therefore, the characteristics of the pre-chosen solvents form an integral part of the cosmetic compositions.

In another preferred embodiment of the present invention, the functional radical of the functionalised polyanhydride is selected from the group consisting of inorganic powders such as silicates (mica, talc, kaolin, etc.) and inorganic pigments (such as titanium dioxide, iron oxides, etc.). More specifically, said functional radical is in a dry state.

By appropriately measuring the ratios between lipophilic, hydrophilic chains and/or active functional molecules, different functionalised polyanhydrides for specific cosmetic applications are obtained.

Specifically, the functionalised polyanhydrides of the present invention may be used very advantageously for decorative cosmetics: for the make-up of lips, as stick or cream; for the make-up of eyes, as eye-shadows in pressed powder, as an emulsion or by casting and mascara; for the make up of the face as pressed powder, emulsion or by casting. Other types of cosmetic applications are not excluded.

Examples from 1 to 6, which are functionalisation reactions of polyanhydrides, examples from 7 to 11, which are embodiments of cosmetic compositions containing the dispersions in a solvent of the functionalised polyanhydrides and example 12, which is an embodiment of a cosmetic composition containing the functionalisation of polyanhydrides with inorganic powders, will be set forth thereafter by way of mere non-limitative example.

All percentages are to be intended as weight percentages.

### EXAMPLE 1

### Functionalisation of the Polyanhydride with Sterol Compounds

The synthesis of the functionalised polyanhydride with β-sitosterol uses the following reagent concentrations:

| | % |
|---|---|
| Phase A | |
| Trimethylol propane triisostearate | 66.8 |
| Octadecene-maleic anhydride copolymer | 8.5 |
| β-sitosterol | 4.3 |
| Phase B | |
| Octyl dodecanol | 20.4 |

The copolymer is dispersed in the trimethylol propane triisostearate at approximately 120° C., β-sitosterol is added to the dispersion and is reacted for 4 hours. In order to complete the reaction, octyl dodecanol is added protracting heating to 110° C. for 1 hour. Finally, it is maintained at 80° C. for a further 12 hours. A functionalised polyanhydride dispersion is obtained having trimethylol propane triisostearate as a dispersing agent.

### EXAMPLE 2

### Functionalisation of the Polyanhydride with Lipids

The synthesis of the functionalised polyanhydride with different chain length lipids uses the concentrations of reagents indicated in the following table:

| | % |
|---|---|
| Phase A | |
| Trimethylol propane triisostearate | 33.5 |
| Octadecene-maleic anhydride copolymer | 30.35 |
| Dodecyl hexadecanol | 9.22 |
| Phase B | |
| Octyl dodecanol | 26.93 |

The copolymer is dispersed in the trimethylol propane triisostearate at approximately 120° C., dodecyl hexadecanol is added to the dispersion and is reacted for 3 hours. In order to complete the reaction, octyl dodecanol is added protracting heating for a further 3 hours. Finally, it is maintained at 80° C. for a further 12 hours. A functionalised polyanhydride dispersion is obtained having trimethylol propane triisostearate as a dispersing agent.

### EXAMPLE 3

### Multifunctionalisation of the Polyanhydride with Lipids Hydrophilic Molecules and Active Compounds

The synthesis of the functionalised polyanhydrides with several substituents having different nature uses the reagent concentrations indicated in the following table:

| | % |
|---|---|
| Phase A | |
| Isododecane | 64.7 |
| Octadecene-maleic anhydride copolymer | 24.0 |
| Dodecyl hexadecanol | 5.0 |
| Phase B | |
| Sorbitantristearate | 5.0 |
| Phytosphingosine | 0.5 |
| Water | 0.6 |
| Citric acid | 0.2 |

The copolymer is dispersed in the isododecane at approximately 110° C., dodecyl hexadecanol is added to the dispersion and is reacted for 3 hours. Sorbitantristearate, phytosphingosine and the catalyst solution is added to water reacting for a further 3 hours. Finally, it is maintained at 80° C. for a further 12 hours. A multifunctionalised polyanhydride dispersion is obtained having isododecane as a dispersing agent.

### EXAMPLE 4

### Functionalisation of the Polyanhydride with Silicone Polymers

The synthesis of the functionalised polyanhydride with the silicone polymer uses the following reagent concentrations:

| | % |
|---|---|
| Phase A | |
| Isododecane | 62.4 |
| Octadecene-maleic anhydride copolymer | 18 |
| Myristic alcohol | 8 |
| Water | 1.3 |
| Citric acid | 0.3 |
| Phase B | |
| Dimethiconol | 10 |

The copolymer is dispersed in the isododecane at approximately 120° C., myristic alcohol and the catalyst solution are added to the dispersion, and it is reacted for 3 hours. Difunctional dimethiconol is added protracting the reaction for a further 3 hours. Finally, it is maintained at 80° C. for a further 12 hours. A functionalised polyanhydride dispersion is obtained having isododecane as a dispersing agent.

### EXAMPLE 5

### Multifunctionalisation of the Polyanhydride with Lipids

| | % |
|---|---|
| Phase A | |
| Isodecane | 61.55 |
| Octadecene-maleic anhydride copolymer | 17 |
| Myristic alcohol | 7 |
| Hexyldecanol | 4 |
| Dimethiconol | 6 |
| Hydrogenated dilinoleyl alcohol | 2 |
| Phytosphingosine | 0.2 |
| Phase B | |
| Water | 1.85 |
| L-Arginine | 0.4 |

The copolymer is dispersed in isododecane. Myristic alcohol, hexyldecanol, dimethiconol, hydrogenated dilinoleyl alcohol are subsequently added to the dispersion which is reacted at a temperature of 120° C. for 18 hours. After these 18 hours, the temperature is taken to 90° C. and the water and arginine solution is added and reacted for two hours.

### EXAMPLE 6

### Functionalisation of Polyanhydrides with Inorganic Powders

| | % |
|---|---|
| Phase A | |
| Octadecene-maleic anhydride copolymer | 1.5 |
| Sodium hydroxide | 0.31 |
| Water | 27.75 |
| Phase B | |
| Talc | 13.74 |
| Mica | 4 |
| Phase C | |
| HCl solution | 2.7 |
| Phase D | |
| Water | 50 |

Phase A is mixed at 75° C. to complete dissolution and then processed at room temperature. Protracting mixing, phase B and phase A are initially added and phase C is subsequently added to said mixture A+B. Phase D is added to the resulting dispersion and the mixing is protracted. The resulting product is finally dried.

### EXAMPLE 7

### Powder Eye-Shadow

Cosmetic composition containing the polyanhydride of Example 1

| Component | % |
|---|---|
| Mica coated with silicone | 33.5 |
| Mica | 28.2 |
| Brown iron oxide | 23.8 |
| Functionalised polyanhydride (ex. 1) | 14.0 |
| Preservatives/Antioxidants | 0.5 |

### EXAMPLE 8

### Lipstick

Cosmetic composition containing a mixture of polyanhydrides of Example 1 and Example 2

| Component | % |
|---|---|
| Octyl dodecanol | 16.8 |
| Microcrystalline wax | 10.5 |
| Functionalised polyanhydride (example 1) | 20.0 |
| Functionalised polyanhydride (example 2) | 25.0 |
| PVPeicosene copolymer | 5.0 |
| Capric/caprylic triglyceride, stearalkonium | 8.0 |
| hectorite, propilen carbonate | |
| Pigments | 14.5 |
| Perfume | 0.1 |
| Preservatives | 0.1 |

### EXAMPLE 9

### Mascara

Cosmetic composition with the polyanhydride of Example 3

| Component | % |
|---|---|
| Water | 28.2 |
| Glycerine | 2.0 |
| Hydroxyethylcellulose | 1.0 |
| Acacia Senegal rubber | 3.0 |
| Water | 3.0 |
| Iron oxides | 10.0 |
| Microcrystalline wax | 6.0 |
| Hydrogenated rapeseed oil | 5.0 |
| Tribehenin | 4.0 |
| Glyceryl stearate | 6.0 |
| Cetearyl glucoside and cetaryl alcohol | 5.0 |
| Multifunctionalised polyanhydride (example 3) | 15.0 |
| Preservatives | 0.6 |
| Ethylene/vinylacetate copolymer | 11.0 |

### EXAMPLE 10

### No Transfer Fluid Lipstick

A cosmetic composition with the polyanhydride of Example 4

| Component | % |
|---|---|
| Silicone functionalised polyanhydride (example 4) | 93.0 |
| Ethanol | 3.0 |
| Pigments | 4.0 |

### EXAMPLE 11

### No Transfer Fluid Lipstick

A cosmetic composition with the polyanhydride of Example 5

| Component | % |
|---|---|
| Multifunctionalised polyanhydride with lipids (example 5) | 91.2 |
| Glossy pigments | 3.0 |
| Pigments | 2.5 |
| Water | 3.0 |
| L-Arginine | 0.2 |
| Perfume | 0.1 |

### EXAMPLE 12

### Face Powder

A cosmetic composition containing the polyanhydride of Example 6

| Component | % |
|---|---|
| Functionalised polyanhydride (ex. 6) | 60.0 |
| Mica | 23.9 |
| Titanium dioxide | 0.5 |
| Yellow iron oxide | 2.0 |
| Red iron oxide | 2.3 |
| Brown iron oxide | 1.8 |
| Nylon 12 | 4.0 |
| Silica | 1.5 |
| Binding agent | 3.5 |
| Preservatives/Antioxidants | 0.5 |

1. A cosmetic composition, comprising a functionalized polymer, which is a functionalized poly(alphaolefin-co-malcic anhydride) copolymer of formula IV having a molecular weight comprised between 500 and 1,000,000 g/mol 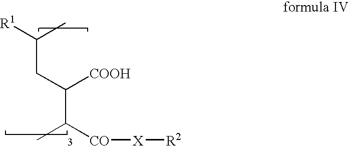
wherein
R¹ is a C₂—C₃₄ alkyl;
—X— is —O— or —NH—;
—R² is a functional radical which contains at least one OH or NH₂ group, though not amine oxides, nor secondary or ternary amines; and
—X—R² together is a radical selected from the group consisting of:
molecules active on the epidermal physiology selected from the group consisting of ceramides, panthenyl ethyl ether, sterols and tocopherols;
lipids selected from the group consisting of linear or branched, saturated or unsaturated C₂—C₃₆ alkyl alcohols, alkyl diols, alkyl amines, alkyl hydroxyl esters, sorbitol esters and saccharose esters;
hydrophilic molecules selected from the group consisting of glycerines, mono and di-esters of glycerine and polyglycerols, amino acids and derivatives thereof, monosaccharides, oligosaccharides, low substitution polysaccharides and ethoxylated alcohols;
silicones selected from the group consisting of molecules, silicone oligomers and polymers and fluorine-derivatives selected from molecules, oligomers and fluorinated polymers;
inorganic powders selected between silicates chosen from mica, talc and kaolin, and inorganic pigments chosen from titanium dioxide and iron oxides; and
natural molecules active in the absorption of UV radiation selected from the group consisting of tannins, flavonoids, thymol, caffeic acid esters and vitamin E,
and solvents, waxes, excipients, dyes and other cosmetic acceptable ingredients;
wherein said functionalized polymer is dispersed in a solvent or in its reaction medium chosen from the group consisting of (C₈—C₂₀) triglycerides; alcohol or polyalcohol esters with fatty acids selected between trimethylol propane triisostearate and octyl stearate; hydrocarbons selected between isododecane and hydrogenated polyolefins; silicones selected between cyclopentasiloxane and dimeticones; and organosilicones.

2. A cosmetic composition according to claim 1, wherein said composition contains from 0.5 wt % to 95 wt % of the dispersion of the functionalized polyanhydride of formula IV in the solvent or in its reaction medium.

3. A cosmetic composition according to claim 1, wherein the concentration of functionalized polymer of formula IV in its reaction medium is comprised between 1 and 85 wt %.

4. A cosmetic composition according to claim 1, wherein the functional radical is selected from the group consisting of molecules active on the epidermal physiology selected from the group consisting of ceramides, panthenyl ethyl ether, sterols and tocopherols.

5. A cosmetic composition according to claim 1, wherein the functional radical is selected from the group consisting of lipids selected from the group consisting of linear or branched, saturated or unsaturated C₂—C₃₆ alkyl alcohols, alkyl diols, alkyl amines, alkyl hydroxyl esters, sorbitol esters, and saccharose esters.

6. A cosmetic composition according to claim 1, wherein the functional radical is selected from the group consisting of hydrophilic molecules selected from the group consisting of glycerine, mono and di-esters of glycerine and polyglycerols, amino acids and their derivatives, monosaccharides, oligosaccharides, low substitution polysaccharides and ethoxylated alcohols.

7. A cosmetic composition according to claim 1, wherein the functional radical is selected from the group consisting of silicones selected from the group consisting of molecules, oligomers and silicone polymers and of fluorine-derivatives selected from molecules, oligomers and fluorinated polymers.

8. A cosmetic composition according to claim 1, wherein the functional radical is selected from the group consisting of inorganic powders selected between silicates chosen from mica, talc and kaolin and inorganic pigments chosen from titanium dioxide and iron oxides.

9. A cosmetic composition according to claim 1, wherein the functional radical is selected from the group consisting of natural molecules active in the absorption of UV radiation selected from the group consisting of tannins, flavonoids, thymol, caffeic acid esters and vitamin E.

10. A cosmetic composition according to claim 1, wherein the functionalized polymer of formula IV is multifunctionalized with two or more different functionalized molecules chosen from the radicals listed in claim 1.

11. A cosmetic composition according to claim 1, wherein said copolymer of formula IV has a molecular weight comprised between 4,000 and 1,000,000 g/mol.

12. A cosmetic composition according to claim 1, wherein said lipids comprise C₂—C₃₄ alcohols.

13. A process to prepare functionalized polymers of formula IV, wherein polyanhydrides of formula V are reacted with functional molecules of formula R²—X—H in a reaction solvent, according to the following scheme: 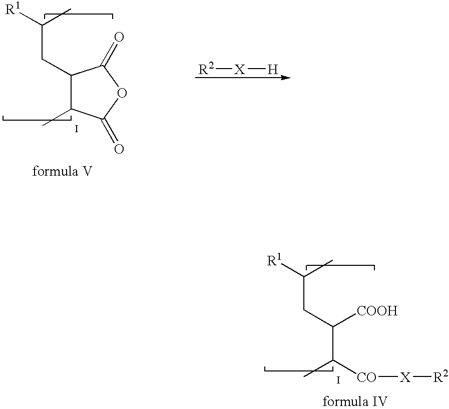
wherein said reaction solvent is chosen from the group consisting of (C₈—C₂₀) triglycerides; alcohol or polyalcohol esters with fatty acids selected between trimethylol propane triisostearate and octyl stearate; hydrocarbons selected between isododecane and hydrogenated polyolefins; silicones selected between cyclopentasiloxane and dimeticones; organosilicones and water.

14. A cosmetic product prepared with a composition or a mixture of two or more cosmetic compositions according to claim 1.

15. A cosmetic product according to claim 14 for decorative cosmetics, comprising a foundation, a powder, an eye-shadow, a lipstick, a mascara, in the form of loose or cake powder, fluid or cream emulsion, or cast.

16-17. (canceled)

18. A cosmetic composition according to claim 2, wherein said composition contains from 10 wt % to 50 wt % of the dispersion of the functionalized polyanhydride of formula IV in the solvent or in its reaction medium.

19. A cosmetic composition according to claim 3, wherein the concentration of functionalized polymer of formula IV in its reaction medium is comprised between 15 to 60 wt %.
